(19) United States
(12) Patent Application Publication
(10) Pub. No.: US 2002/0177574 A1
(43) Pub. Date: Nov. 28, 2002

(54) Endometrial gene therapy

(76) Inventor: Hugh S. Taylor, Shelton, CT (US)

Correspondence Address: Barry L. Kelmachter, BACHMAN & LaPOINTE, P.C., 900 Chapel Street, Suite 1201, New Haven, CT, 06510-2802, US

(21) Appl. No.: 10/127,219

(22) Filed: Apr. 19, 2002

Related U.S. Application Data
(60) Provisional application No. 60/285,102, filed on Apr. 20, 2001.

(51) Int. Cl. A61K 48/00; A61K 9/127

(52) U.S. Cl. 514/44; 424/450

(57) ABSTRACT

A method for treating the uterus includes transfecting the uterus with a liposome-mediated gene. Hox genes have been found particularly relevant to diseases such as endometriosis, and manipulation of their expression can affect embryonic implantation.

### CROSS-REFERENCE TO RELATED APPLICATION(S)

This application claims the benefit of U.S. Provisional Patent Application Serial No. 60/285,102, filed Apr. 19, 2001, entitled ENDOMETRIAL GENE THERAPY.

### BACKGROUND OF THE INVENTION

The invention relates to endometrial gene therapy.

Endometriosis affects at least 10% of reproductive age women and is characterized by the presence of ectopic endometrium. The association between endometriosis and infertility is well established, but the mechanisms responsible are unknown.

It is therefore the primary object of the present invention to provide therapy or treatment to address infertility due to endometriosis.

It is a further object of the present invention to provide improvement in embryonic implantation and development.

It is a still further object of the present invention to provide treatment and therapy which can be utilized in a contraceptive manner, as well.

Other objects and advantages will appear hereinbelow.

### SUMMARY OF THE INVENTION

In accordance with the present invention, the foregoing objects and advantages have been readily attained.

According to the invention, gene therapy is used for treating the uterus and can advantageously be used to alter endometrial receptivity, which has application in the fields of infertility and contraception.

The Hox gene and expression of this gene have been found to be critical to embryonic implantation and development, and suppression of this gene can lead to infertility, providing for contraceptive use of the present invention if desired.

In addition, it has been found that liposome mediated gene therapy to the uterus is safe, effective, and provides for reproducible results.

### BRIEF DESCRIPTION OF THE DRAWINGS

A detailed description of preferred embodiments of the present invention follows, with reference to the attached drawings, wherein:

FIG. 1 is a representative sample of Ishikawa cells treated with X-gal following cationic liposome-mediated transfection with pcDNA3.1(+)/LacZ, and showing that more than 50% of cells are stained intensely blue and a further 30% demonstrate moderate blue staining;

FIG. 2 is a representative sample showing that none of the Ishikawa cells transfected with controlled DNA (pcDNA3.1(+)) demonstrated any blue staining following treatment with X-gal;

FIG. 3 illustrates sections of murine endometrium treated with X-gal following cationic liposome-mediated transfection with pcDNA3.1(+)/LacZ, wherein nearly 100% of glandular and approximately 10% of stroma cells are stained blue;

FIG. 4 shows sections of uterus transfected with control (pcDNA3.1(+)) and subsequently stained with X-gal, which demonstrate minimal or absent staining;

FIG. 5 illustrates Northern analysis of Hoxa10 expression in Ishikawa cells transfected with either a Hoxa10 antisense oligonucleotide or pcDNA3.1(+)/HOXA10, and shows no change in Hoxa10 mRNA in Ishikawa cells transfected with the Hoxa10 antisense oligonucleotide compared with Hoxa10 mRNA from cells transfected with a control missense oligonucleotide;

FIG. 6 shows Western analysis performed on cells transfected with a Hoxa10 antisense oligonucleotide and demonstrates a decrease in Hoxa10 protein compared with control missense transfected cells, and an increase in Hoxa10 protein as compared with pcDNA3.1 transfected controls for cells transfected with pcDNA3.1(+)/HOXA10;

FIG. 7 illustrates alterations in implantation following liposome-mediated gene transfection in vivo with a Hoxa10 antisense oligonucleotide, and shows that the average number of implantation sites was 13.3 in mice transfected with a control missense oligonucleotide, and 6.5 in mice transfected with the Hoxa10 antisense oligonucleotide;

FIG. 8 illustrates alterations in birth rate following liposome-mediated gene transfection in vivo with pcDNA3.1-HOXA10, wherein in the control pcDNA3.1(+) transfected group, litter size ranged from 0 to 13, and in the pcDNA3.1-HOXA10 group, mice gave birth to between 11 and 14 pups;

FIG. 9 illustrates HOXA10 expression in ectopic endometrium of patients with endometriosis as demonstrated by Northern blot analysis, wherein expression is seen in the first and second half of the proliferative phase (P1 and P2, respectively) of the menstrual cycle after hybridization to a probe specific to the HOXA10 or G3PD genes, and wherein representative samples showed a lack of significant up-regulation between the proliferative phase (P1 and P2) and the mid- and late secretory phase (S2 and S3);

FIG. 10 illustrates HOXA10 expression in the endometrium of women with and without endometriosis;

FIG. 11 illustrates HOXA11 expression in the endometrium of patients with and without endometriosis, and controls showed the expected increase in HOXA11 at the time of implantation;

FIG. 12 illustrates low power (100×) photomicrographs of the human uterus transfected with pcDNA3.1/LacZ, wherein the top panel illustrates immunohistochemistry performed with a mouse monoclonal antibody demonstrating successful expression of β-Galactosidase in endometrial glands, stroma and myometrium, with control uterus transfected with pcDNA3.1, and wherein the bottom panel shows transfection with pcDNA3.1/LacZ and pcDNA3.1 (control), each done on two uteri;

FIG. 13 shows high power photomicrograph (400×) showing the results of uteri transfected with LacZ (left) and corresponding controls (right), wherein endometrial glands demonstrating high levels of β-galactosidase expression in 100% glandular epithelial cells, endometrial Stroma demonstrated β-galactosidase expression in all cells, with intense staining seen in 20% of cells and moderate staining observed in 50% of cells, and wherein the myometrium (0.25 cm from the endometrial-myometrial junction) demonstrated intense staining in 15% of cells and light staining in 60% of cells;

FIG. 14 shows quantification of immunohistochemistry results by H-Score, wherein intensity of staining was graded on the basis of an arbitrary scale from 0 to 3 characterized as no staining (0), weak but detectable (1), distinct (2) and very strong (3), and shows H-Score values between the LacZ treated and control group for each cell type, when analyzed using the Mann-Whitney Test are significant at p<0.0001, and wherein scores between the cell types in the LacZ treated uterus were compared using ANOVA and each was significantly different from each of the others at p<0.0001; and

FIG. 15 is a high power (400×) photomicrograph of sub-serosal myometrium beyond 1.8 cm from the endometrial-myometrial junction, and demonstrates no significant staining, transfection limited to the endometrium and proximal myometrium, and that vasculture does not express β-galactosidase indicating that transfection was limited to the target tissues.

### DETAILED DESCRIPTION OF THE PREFERRED EMBODIMENT(S)

Mice with a targeted mutation of the Hoxa10 gene demonstrate uterine factor infertility. It is unclear if the defect in the uterine environment arises due to the absence of Hoxa10 expression during embryonic development or in the adult. We have recently demonstrated that HOXA10 expression in human endometrium rises dramatically at the time of implantation, suggesting maternal expression of Hoxa10/HOXA10 may be essential to the process. To assess the importance of maternal Hoxa10 expression, the uteri of day 2 pregnant mice were injected with a DNA/liposome complex containing constructs designed to alter maternal Hoxa10 expression before implantation. Transfection with a Hoxa10 antisense oligodeoxyribonucleotide significantly decreased the number of implantation sites. Transfection with a plasmid which constitutively expresses Hoxa10 optimized several of implanted embryos resulting in increased litter size. These results demonstrate that maternal Hoxa10 expression is essential for implantation and is the first report of the maternal alteration of a gene known to affect implantation specifically. We also demonstrate that DNA/liposome complexes containing the same Hoxa10 constructs that alter fertility in mice, can affect Hoxa10 expression in a human endometrial cell line. Alteration of human endometrial HOXA10 via liposome-mediated gene transfection is a potential contraceptive agent or fertility treatment.

The molecular mechanisms responsible for appropriate endometrial development and receptivity to implantation are poorly understood. Hox genes are leading candidates for regulating differentiation of the endometrium in preparation for embryonic implantation. Hox genes are transcriptional regulators that have an important role in embryonic development. They assign the correct identity to undifferentiated body segments along several developmental axes. Four genes of the Hoxa cluster, Hoxa9, Hoxa10, Hoxa11 and Hoxa13 are typically expressed in the developing reproductive system of the mouse. Hox genes are typically expressed only during embryogenesis and their role in this process has been well characterized. However, we have previously shown that Hoxa genes demonstrate persistent adult expression in both the mouse and human female reproductive tract. It is possible that the continued expression of these Hoxa genes allows the reproductive tract to maintain developmental plasticity and to differentiate appropriately during each menstrual cycle and throughout pregnancy.

Female mice with a targeted mutation of Hoxa10 demonstrate uterine factor infertility. These mice ovulate normally and produce normal embryos. However, the uteri of Hoxa10-deficient mice will not support the implantation of either their own or wild-type embryos. Hoxa10/HOXA10 is expressed in both the embryonic and adult endometrium of mice and humans. In humans, we have previously demonstrated a dynamic expression pattern of HOXA10 in the endometrium throughout the menstrual cycle; HOXA10 expression rises significantly in the mid-secretory phase, the time of implantation. The endometrium undergoes complex structural and functional changes during each menstrual cycle to allow successful implantation and subsequently to support the embryo through effective placentation. It has been proposed that the implantation defect seen in Hoxa10-deficient mice is due to an abnormality in the embryonic development of the reproductive tract. However, the continued expression of HOXA10 in the adult endometrium may regulate the cyclic structural and functional changes of this tissue in a manner analogous to embryonic development. The menstrual cycle-specific pattern of HOXA10 in human endometrium suggests that maternal Hoxa10/HOXA10 may be necessary for implantation.

Here we demonstrate that maternal Hoxa10 expression in the endometrium is essential for implantation. We use liposome-mediated gene transfection to either block maternal Hoxa10 expression with a Hoxa10 antisense oligonucleotide or to increase maternal Hoxa10 expression with a plasmid that constitutively expresses Hoxa10. These experiments demonstrate that manipulation of adult Hoxa10 expression significantly affects fertility.

This is the first time that gene transfection of the adult uterine endometrium has been shown to affect successfully a gene necessary for implantation. We have previously shown that, based on the rise in HOXA10 expression at the time of implantation, maternal HOXA10 is probably similarly required for this process in women. The alteration of human HOXA10 expression in the adult endometrium is a potential contraceptive agent or fertility treatment.

The ability of a DNA/liposome complex to transfect endometrial cells was demonstrated by transfecting both Ishikawa cells and mouse endometrium in vivo with pcDNA3.1(+)/LacZ.

Ishikawa cells are a well differentiated cell line that express estrogen and progesterone receptors, and are the best available model of endometrium.

Ishikawa cells were transfected for 5 h with pcDNA3.1(+)/LacZ. The cells were maintained in culture for a further 19 h, fixed and then stained with X-gal for 1 h. More than 50% of cells stained intensely blue and a further 30% demonstrated moderate blue staining. None of the cells transfected with control DNA (pcDNA3.1(+) without the LacZ gene) showed by blue staining. Representative samples are shown in FIGS. 1 and 2.

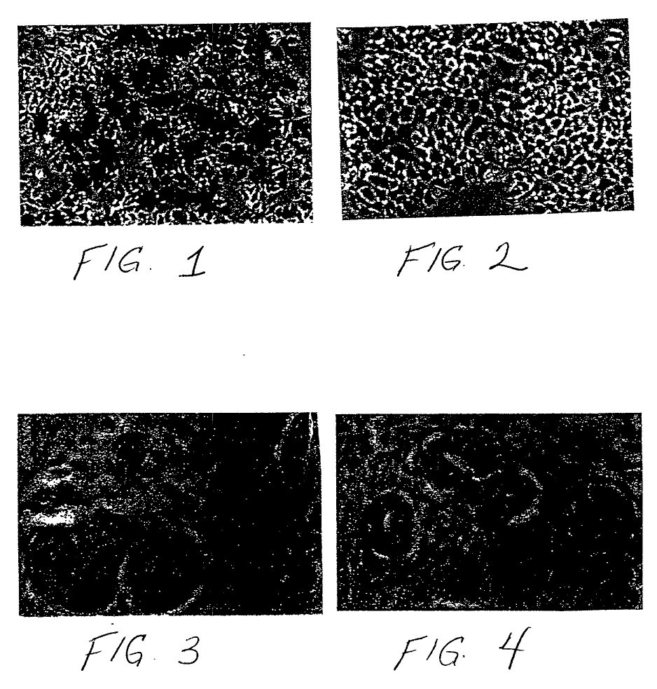

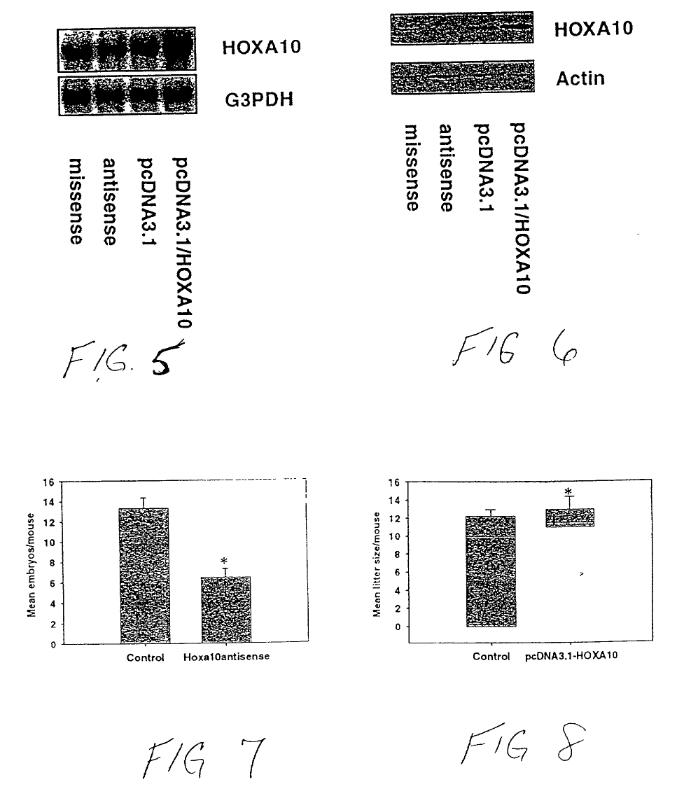

To demonstrate the endometrium could also be transfected in vivo, a DNA/liposome complex containing pcDNA3.1(+)/LacZ was injected into the lumen of each uterine horn. Forty-eight hours later, the time of implantation, the uteri were sectioned and stained with X-gal for 24 h. FIG. 3 shows murine endometrium transfected with pcDNA3.1(+)/LacZ and subsequently stained with X-gal. One hundred percent of glandular cells and 10% of stromal cells stained blue. Sections of mouse uterus transfected with control DNA and then treated with X-gal demonstrated minimal or absent staining (FIG. 4). Serial sections from proximal to distal uterus revealed uniform results.

To determine that Hoxa10 expression can be manipulated in endometrial cells using a Hoxa10 antisense oligodeoxyribonucleotide, Ishikawa cells were transfected with a DNA/liposome complex containing a Hoxa10 antisense oligonucleotide, complementary to the translation start site of Hoxa10. This sequence is conserved in the mouse and human. A cell culture model was used due to the small amount of endometrial tissue per mouse and relatively low abundance of Hoxa10 protein that make these experiments impractical in vivo. HOX gene expression in Ishikawa cells is well characterized and has been demonstrated to be regulated as in the endometrium. Following transfection, cellular RNA and protein were extracted and Northern and Western analyses performed.

Levels of Hoxa10 mRNA were compared between cells transfected with either a Hoxa10 antisense oligonucleotide or a missense control oligonucleotide using Northern blot analysis. The values obtained for Hoxa10 were normalized to G3PDH. No change in Hoxa10 mRNA was seen in Ishikawa cells transfected with the Hoxa10 antisense oligonucleotide compared with Hoxa10 mRNA in cells transfected with a control oligonucleotide, suggesting normal transcription of Hoxa10 in the presence of the Hoxa10 antisense oligonucleotide (FIG. 5).

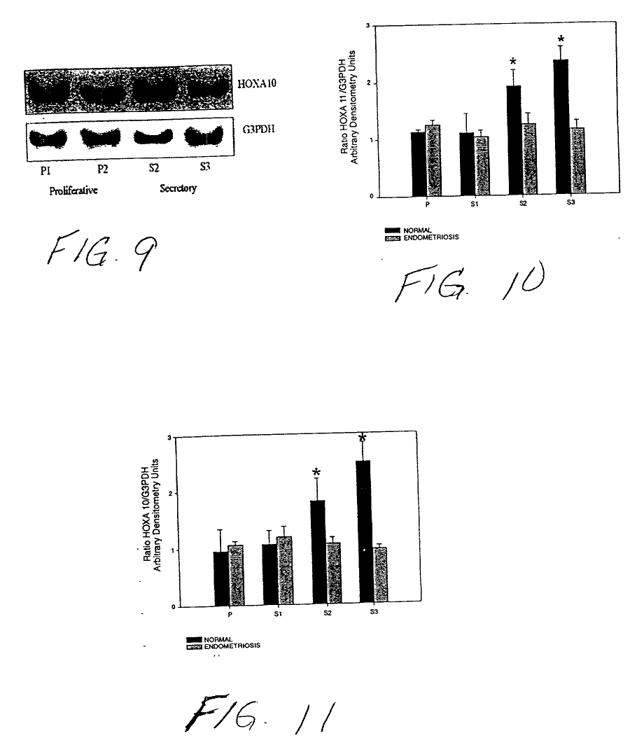

Levels of Hoxa10 protein were decreased in cells transfected with the Hoxa10 antisense oligonucleotide to approximately 15% of the level expressed in cells receiving the control oligonucleotide. Representative lanes from Western analysis are shown in FIG. 6, suggesting decreased translation of Hoxa10.

To demonstrate that Hoxa10 expression can be manipulated in endometrial cells, Ishikawa cells were transfected with pcDNA3.1(+)/HOXA10 or pcDNA3.1(+), containing no insert, as a control. Levels of Hoxa10 mRNA and protein were compared between these two groups.

Ishikawa cells transfected with pcDNA3.1(+)/HOXA10, demonstrated an increase in Hoxa10 mRNA compared with cells transfected with pcDNA3.1(+) (FIG. 5). The increase in mRNA persisted for at least 24 h. An increase was also observed in Hoxa10 protein in cells transfected with pcDNA3.1(+)/HOXA10 compared with cells transfected with pcDNA3.1(+), as demonstrated in FIG. 6.

In order to block the maternal expression of Hoxa10 in mice that have normal Hoxa10 expression during embryogenesis, the antisense oligonucleotide previously described was used to transfect the endometrium of mice. A DNA/liposome complex containing the Hoxa10 antisense oligonucleotide was injected into the base of each uterine lumen 30-60 h following detection of a vaginal plug. A total of 34 mice received either the antisense or the control missense oligonucleotide.

At day 9 of pregnancy, mice were killed and the uteri examined for implantation sites. The average number of implantation sites was 13.3 in 13 mice transfected with the control missense oligonucleotide and 6.5 in the 20 mice transfected with the antisense oligonucleotide (FIG. 7). A two-tailed unpaired t test demonstrated a significant difference between the average number of implantation sites in the two groups (P=0.00002). In the 30 mice treated with the Hoxa10 antisense oligonucleotide, there was no significant difference between the number of implantation sites seen at day 9 in 18 mice and live births in 12 mice.

The endometrium appeared normal histologically, demonstrating adequate decidualization. Pups of mice transfected with Hoxa10 antisense were born following a normal length gestation of 17-20 days, were of normal size and demonstrated no morphological abnormalities. Male and female pups of mice transfected with the Hoxa10 antisense oligonucleotide mated with wild-type mice and demonstrated normal fertility and litter size.

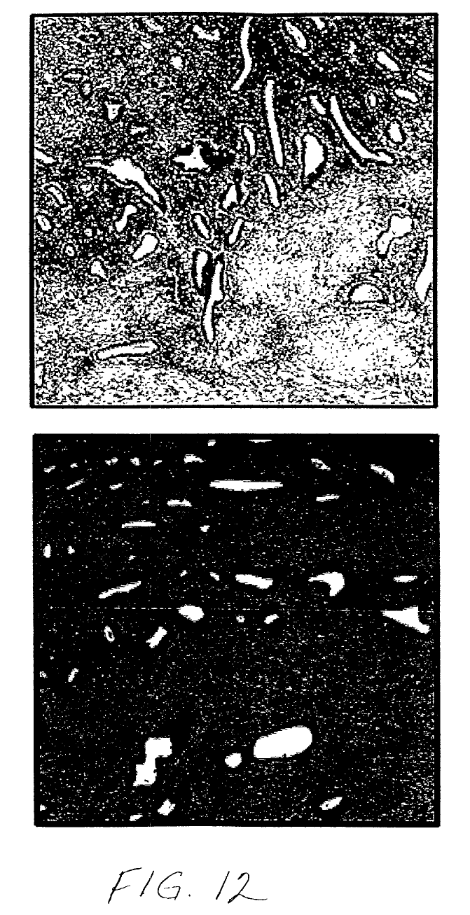

To increase adult expression of Hoxa10, the endometrium of day 2 pregnant mice were transfected with either pcDNA3.1(+)/HOXA10 or pcDNA3.1(+) as a control. At day 9 of pregnancy, there was no significant difference between the average number of implantation sites in mice transfected with pcDNA3.1(+)/HOXA10 compared with controls transfected with pcDNA3.1(+) (data not shown). However, live birth rates did vary. Litter size ranged from 0 to 13 in nine pcDNA3.1(+) transfected controls whereas the 10 mice transfected with pcDNA3.1(+)/HOXA10 gave birth to between 11 and 14 pups (FIG. 8). The HOXA10-treated mice gave birth to larger litters with less variability than the controls. The difference between the average litter size in each group was demonstrated to be significant using a two-tailed unpaired t test (P=0.024).

The implantation sites appeared histologically normal. There was no significant difference in placental weights or vasculature. Pups of mice transfected with pcDNA3.1(+)/HOXA10 were born following a normal length gestation of 17-20 days, were of normal size and demonstrated no morphological abnormalities. Male and female pups of mice transfected with pcDNA3.1(+)/HOXA10 mated with wild-type mice and demonstrated normal fertility and litter size.

The role of Hox genes in embryonic development is well-established. However, the functions of Hox genes in the adult are poorly characterized. Based on this paradigm, Hoxa10 was presumed to function during the embryogenesis of the reproductive tract. However, indirect evidence has suggested a role for maternal Hoxa10 expression in implantation. We have previously shown that four genes of the HOXA cluster demonstrate continued expression in the adult reproductive tract. Furthermore, we have shown that HOXA10 is expressed in the adult endometrium in a dynamic way that varies with the menstrual cycle suggesting a role for maternal HOXA10 in implantation.

Our present work shows that maternal expression of Hoxa10 in the endometrium of mice is essential for optimal implantation. This is the first report of the maternal alteration of a gene known to affect specifically implantation. When mouse endometrium is transfected with a Hoxa10 antisense oligonucleotide at the time of implantation, the number of embryos which implant in the uterus are significantly reduced compared with controls. The significant decrease in implantation sites in the uterus transfected with a Hoxa10 antisense oligonucleotide demonstrates that, in the background of normal embryonic Hoxa10, optimal implantation requires maternal Hoxa10 expression.

Fertility in mice can also be affected by increasing Hoxa10 expression. When mouse endometrium is transfected with HOXA10 cDNA, live birth rates increase compared with controls, despite the fact that implantation rates are unaltered, β-Galactosidase activity can still be detected on day 18 of pregnancy following liposome-mediated gene transfection with pcDNA3.1(+)/LacZ, suggesting that Hoxa10 expression from pcDNA3.1(+)/HOXA10 is likely to be present in the endometrium of pregnant mice at least until parturition. The continued expression of increased levels of Hoxa10 in the pregnant uterus may improve placentation and hence optimize the ability of each implanted embryo to survive to term. While litter size is limited by the number of oocytes produced, consistently larger litters are seen with increased Hoxa10 expression; embryo loss may be a major determinant of litter size in controls. We have identified a novel role of Hoxa10 in the maintenance of pregnancy.

We expected that the transfection of the uterine lumen approximately 24 h before the embryo entered to uterine horns would result in an effect on the endometrium and not the embryo directly. Supporting this, pups of mice transfected with either a Hoxa10 antisense oligonucleotide or pcDNA3.1(+)/HOXA10 were born following a normal length gestation of 17-20 days. Pups were of normal size, demonstrated no morphological abnormalities and the placentas from both groups were normal. Male and female pups from both groups mated with wild-type mice. These matings resulted in viable offspring and normal litter size. Taken together, this demonstrates that the effect on pregnancy rates following injection of DNA/liposome complexes into the uterine lumen is probably due to the specific effect of gene transfection of the endometrium only and not an effect on the embryo.

Few tissues have been identified which demonstrate persistent Hox gene expression in the adult. Adult Hox gene expression has been shown to occur in tissues which continue to differentiate in the adult. The role of Hox genes in the structural and functional differentiation of adult tissues is probably analogous to that in embryonic developmental processes. The endometrium undergoes complex structural and functional changes during each menstrual cycle in preparation for implantation and subsequent pregnancy. The alteration of maternal expression patterns of Hoxa10 affecting the function of the endometrium provides further evidence that adult Hox gene expression is important in tissues that undergo continued differentiation past the embryonic period. It is probable that the persistent expression of Hoxa10 in the adult enables the endometrium to retain developmental plasticity and allows the sequential differentiation of the endometrium during each menstrual cycle.

The liposome-mediated gene transfection techniques used in this work have identified the functional importance of maternal expression of Hoxa10 in the endometrium. This type of gene transfection allows the alteration of adult gene expression against a background of normal embryonic gene expression. In vivo gene transfection techniques are a potential means to distinguish between the effects of embryonic and adult gene expression on tissue-specific phenotypes. Furthermore, targeted mutations in mice often reduce viability and hence limit experimentation. Gene transfection in the adult is a method that selectively alters gene expression in the tissues in interest and circumvents effects on other tissues that are potentially lethal.

Differential cellular transfection also enables cell-specific gene function to be identified. In this study, we transfected nearly 100% of epithelial glandular cells but only 10% of stromal cells. The glandular expression of Hoxa10 is therefore more likely to be necessary for implantation. Endometrial glandular growth is estrogen dependent, yet glandular estrogen receptors are down-regulated and undetectable at the time of implantation. Inductive stromal-epithelial interactions regulate endometrial growth and differentiation leading to endometrial receptivity. Estrogen presumably stimulates endometrial glandular development via a paracrine mechanism acting through the stromal estrogen receptor. This suggests that glandular Hoxa10 may be one target of paracrine regulation by endometrial stroma. The molecular nature of this mechanism remains to be elucidated.

As a model of human endometrium, we transfected Ishikawa cells, a well differentiated human endometrial adenocarcinoma cell line, with identical constructs to those used in mice. The anti-sense oligonucleotide was complementary to a region conversed in mouse and human Hoxa10/HOXA10. The expression of HOXA10 in Ishikawa cells is well characterized and provides an accurate representation of HOXA10 expression in the human endometrium in vivo. We demonstrated that the constructs that transfect murine endometrium and alter fertility have the ability to alter HOXA10 expression in a human endometrial cell line.

We have previously shown that in the female reproductive tract, Hox gene expression patterns are identical between mouse and human. In view of the necessary role of maternal Hoxa10 expression in mouse implantation, it is likely that the menstrual cycle-specific expression pattern of HOXA10 in women is similarly required for implantation. Genetic manipulation in humans using gene transfection is a developing technique, which has wide reaching therapeutic potential. Furthermore, cationic liposomes have been shown to be an effective and efficient method for delivery of genetic material to human tissue in vivo. Similar gene transfection techniques in human endometrium designed to manipulate HOXA10 expression may allow improved control of human implantation and reproduction. Manipulation of human expression patterns of HOXA10 in the adult endometrium may have potential as a contraceptive or fertility treatment.

HOXA10 cDNA (a generous gift from C Largman, University of California Calif., USA) was cloned into the EcoRI site of pcDNA3.1(+) (Invitrogen, Carlsbad, Calif., USA) pcDNA3.1(+) without the HOXA10 insert and pcDNA3.1(+)/LacZ were used as controls (Invitrogen).

Two 30 mer phosphothiorate oligodeoxyribonucleotides were synthesized at the WM Keck Oligonucleotide Laboratory at Yale University; one complementary to the start of translation of Hoxa10/HOXA10 corresponding to nucleotides 44-73 (GenBank accession number L08757), 5′-CTCTCCGAGCATGACATTGTTGTGGGATAA-3′, the other with the same nucleotide composition but a random sequence was used as a control, 5′-TGCTGCTAGGATCGTTCAAGTGTATCACGA-3′.

For in vitro use, 12 μl DNA was mixed with 300 μl Opti-MEM Reduced Serum Medium (Life Technologies, Gaithersburg, Md., USA), added to 30 μl of 22° C. liposome (a 3:1 (w/w) formulation of 2,3-dioleyloxy-N-[2(sperminecaboxamido)ethyl]-N-N-dimethyl-1-propanaminium trifluoroacetate (DOPSA) and dioleoylphosphatidyl ethanolamine (DOPE) (Life Technologies) in 300 μl 37° C. Opti-MEM and incubated for 45 min at room temperature. A final concentration of 4 μg/ml DNA and 20 μg/ml liposome was obtained by dilution with Opti-MEM to a total volume of 3 ml.

For in vivo use, 2 μl of DNA was mixed with 10 μl of liposome and incubated for 15 min at room temperature. A final concentration of 16 μg/ml DNA and 40 μg/ml liposome was obtained by dilution with 1× Dulbecco's phosphate-buffered saline (PBS) (Life Technologies) to a total volume of 100 μl.

These concentrations were determined in vitro by optimization of the repression of HOXA10 protein expression which was determined by does response experiments. Initially, 0-20 μg of DNA solution was used and 0-50 μl of liposome. Western analysis was used to determine optimal HOXA10 repression.

Ishikawa cells, a well differentiated human endometrial adenocarcinoma cell line (a generous gift from R Hochberg, Yale University, New Haven, Conn., USA) were grown to 80% confluence in Minimum Essential Media with Earles Salts and L-glutamine (MEM) (Life Technologies), 10% fetal bovine serum, 1% sodium pyruvate, 1% penicillin and 1% streptomycin. A 25 cm² cellular monolayer was transfected with 3 ml of a DNA/liposome complex for 5 h, washed in PBS and maintained in MEM as above, for an additional 19 h. For RNA extraction, cells were lysed in 1 ml Trizol (Life Technologies). For protein extraction, cells were lysed with 0.5 ml chilled (0° C.) single detergent lysis buffer (5 mM TrisCl (pH 8.0), 150 mM NaCl, 0.02% sodium azide, 100 μg/ml PMSF, 1 μg leupeptin, 1% Triton X-100). For X-gal staining, cells were fixed with 3 ml 2% formaldehyde, 0.2% glutaraldehyde in PBS at room temperature.

A 128 bp sequence of the 3′ untranslated region of HOXA10 was cloned into the EcoRI and BamHI sites of pGEM3-Z (Promega, Madison, Wis., USA). The construct was linearized with HindIII, ethanol precipitated and used as a template for riboprobe synthesis. Radiolabeled RNA probes were produced by in vitro transcription using a riboprobe kit (Promega), T7-RNA polymerase, and P UTP (Amersham, Arlington Heights, Ill., USA). A human 1.1 kb glyceraldehyde-3-phosphate dehydrogenase (G3PDH) cDNA template (Clontech, Palo Alto, Calif., USA) was used to generate a control probe by in vitro transcription as above.

Ishikawa cells that had been transfected with a DNA/liposome complex containing either a Hoxa10 antisense oligonucleotide or pcDNA3.1(+)/HOXA10 were homogenized with 3 ml Trizol. Total RNA was extracted and size fractionated on a 1% agarose/0.66 M formaldehyde gel and transferred to a nylon membrane. Membranes were hybridized with a P-labeled HOXA10 riboprobe. Hybridization was performed overnight at 60° C. in 50% formamide, 1× SSC, 5× Denhardt's solution and 0.2% tRNA using 2×10⁶ c.p.m./ml of the P-labeled HOXA10 riboprobe. The membrane was washed twice at 68° C. for 30 min in 0.1× SSC and 0.1% SDS. X-Omat AR film (Eastman Kodak, Rochester, N.Y., USA) was exposed overnight at −70° C. The autoradiographic bands were quantified using densitometry. Each HOXA10 band was normalized to the value obtained from the same lane hybridized with G3PDH.

Ishikawa cells, transfected as above, were lysed in single detergent lysis buffer and centrifuged at 12000 g for 2 min at 4° C. The supernatant was loaded onto a 6% SDS polyacrylamide gel, size fractioned and transferred to a nitrocellulose membrane. The membrane was immersed in a 3% gelatin-Tris buffered saline (TBS) (20 mM Tris, 500 mM NaCl) blocking solution for 30 min at room temperature, washed for 10 min in TBS (20 mM Tris, 500 mM NaCl, 0.05% Tween-20, pH 7.5) and then incubated for 1 h with a 1:1000 dilution of HOXA10 polyclonal antibody (BabCo, Richmond, Calif., USA). The membrane was washed with TBS for 5 min at room temperature and incubated for 1 h with a 1:200 dilution of goat anti-mouse IgG-HRP (BioRad, Hercules, Calif., USA). The membrane was then washed 2× in TBS for 5 min at room temperature and immersed in a horseradish peroxidase color developer buffer (BioRad) for 30 min. Photographs were taken immediately following color development.

Nulliparous reproductive age female and male CD1 mice were obtained from Charles River (Wilmington, Mass., USA). Female mice were mated and examined every 8 h until detection of a vaginal plug. The presence of a vaginal plug was designated day 1 of pregnancy. The mice were anesthetized 30-60 h following plug detection with 250 μl of a 5% xylazine/10% ketamine mixture given by intraperitoneal injection. A laparotomy was performed to expose the uterus and 25 μl of the DNA/liposome complex was injected into the base of each uterine horn using a 27-gauge needle. The incision was closed in two layers (peritoneal and cutaneous) with 4-0 vicryl suture. These experiments were conducted in accordance with an approved protocol issued by the Yale Animal Care and Use Committee.

Female mice which received pcDNA3.1(+)/LacZ were killed on day 4 of pregnancy, uteri removed, fixed in 1.25% glutaraldehyde in 20 ml PBS for 30 min, rinsed twice in 20 ml PBS and placed in X-gal staining solution (PBS containing 2 mM MgCl₂, 0.02% NP-40, 0.01% sodium deoxylate, 5 mM K₃Fe (CN) 6, 5 mM K4 (CN) 6 and 1 mg/ml X-gal) for 24 h at room temperature. Tissue was embedded in paraffin, sections obtained and examined by light microscopy.

Fixed Ishikawa cells that had been transfected with pcDNA3.1(+)/LacZ were washed three times with 5 ml PBS and stained with 2 ml of β-gal staining solution (Boehringer Mannheim, Indianapolis, Ind., USA) for 1 h at 37° C. The cells were examined by light microscopy.

Pregnant mice were either killed by cervical dislocation on day 9 of pregnancy or delivered at term. The uterus of each mouse killed on day 9 was dissected from the abdomen and the implantation sites counted using a dissecting microscope. At term, the numbers of pups born were counted on the day of parturition.

It has been found, according to the invention, that HOX gene expression is altered in the endometrium of women with endometriosis.

HOXA10 and HOXA11 are homeobox genes that function as transcription factors essential to embryonic development. We have recently described a role for each of these two genes in regulating endometrial development in the adult during the course a menstrual cycle. Both Hoxa10 and Hoxa11 are essential for implantation in the mouse and appear to play a similar role in women. To investigate the role of HOX genes in the endometrium of women with endometriosis, quantitative Northern blot analysis was performed on the endometrium of 40 normal cycling controls and 40 patients with documented endometriosis. Patients with endometriosis failed to show the expected mid-luteal rise in HOX gene expression as demonstrated in the controls. Aberrant HOX gene expression suggests that altered development of the endometrium at the molecular level may contribute to the aetiology of infertility in patients with endometriosis.

Endometriosis affects at least 10% of reproductive age women and is characterized by the presence of ectopic endometrium. The association between endometriosis and infertility is well established, but the mechanisms responsible are unknown (Olive and Schwartz, 1993). Multiple factors have been implicated including distortion of the pelvic anatomy, abnormalities of hormone secretion (Ayers et al., 1987), alterations in peritoneal fluid (Halme et al., 1987), disorders of fertilization (Mills et al., 1992), and immunoregulatory dysfunction (Witz et al., 1994). Alterations in endometrial development in patients with endometriosis may contribute to endometriosis related infertility. Reports from several in-vitro fertilization (IVF)/embryo transfer programs indicate patients with endometriosis have decreased implantation rates (Hahn et al., 1986; Simon et al., 1994; Arici et al., 1996). A surgically-induced mouse model of endometriosis also demonstrates failure of implantation as a mechanism of endometriosis associated infertility (Hahn et al., 1986). Although histologically normal, examination of eutopic endometrium from women with endometriosis has revealed other defects. Ultrastructural defects have been reported in the endometrium of women with endometriosis (Fedele et al., 1990). Molecular markers of endometrial receptivity are altered in patients with endometriosis; integrin expression patterns are aberrant in the native endometrium of women with endometriosis (Lessey et al., 1995, 1997; Ota and Tanaka, 1997; Hii and Rogers, 1998). Other alterations of biochemical or molecular markers have been noted including metalloproteinases (Osteen et al., 1996; Sharpe Timms, 1997), soluble urokinase-type plasminogen activator (suPA-R) Sillem et al., 1997), oestogen receptor (ER) splice variant (Fujimoto et al., 1997), vascular endothelial growth factor (VEGF) (Shifren et al., 1996), complement (C3) (Bartosik et al., 1987; Isaacson et al., 1990), aromatase (Noble et al., 1996), CA-125 (McBean and Brunstead, 1993), heat shock protein (Ota et al., 1997) and interleukin 6 (IL6) (Tseng et al., 1996).

HOXA10 and HOXA11 are homeobox genes that mediate embryonic development (Krumlauf, 1992; McGinnis and Krumlauf, 1992) including the development of the reproductive tract (Favier and Dolle, 1997; Taylor et al., 1997). They are translated into transcription factors that regulate a battery of downstream genes necessary for growth and differentiation. We have recently demonstrated that HOX genes play an analogous role in endometrial development during the adult menstrual cycle (Taylor et al., 1998, 1999). HOX gene expression possibly regulates the growth and development of the human endometrium (Taylor et al., 1997). HOXA10 and HOXA11 gene expression varies in response to sex steroids during the menstrual cycle, with dramatic up-regulation in the mid-secretory phase, the time of implantation.

Expression of each of these Hox genes is necessary for implantation in the mouse. Mice with a targeted mutation of either of these genes have uterine factor infertility, producing normal embryos, but with a uterus which lacks the ability of wild-type embryos to implant (Hsieh-Li et al., 1995; Satokata et al., 1995; Favier and Dolle, 1997). We have recently shown that HOXA10 and HOXA11 likely play a similar role in human implantation (Taylor et al., 1997, 1998, 1999). HOXA10 and HOXA11 are expressed in the adult human endometrial stroma and glands and are differentially expressed in the developing endometrium during the menstrual cycle. HOX genes may affect endometrial development in a way analogous to their role in embryonic development, leading to endometrial growth, differentiation and receptivity. In women HOXA10 and HOXA11 are up-regulated in the mid-secretory endometrium, at the time of implantation. The extraordinarily high conservation of HOX gene function and the spatial and temporal expression pattern of HOXA10 and HOXA11 in the endometrium suggest that these HOX genes play an essential role in human implantation. In this study we determined the levels of expression of HOXA10 and HOXA11 in the eutopic endometrium of patients with endometriosis. Alterations of the HOXA10 and HOXA11 genes, whose expression is necessary for implantation, may provide evidence of molecular alterations in the endometrium of these patients, and would suggest a defect in the development and receptivity of the endometrium in patients with endometriosis.

Endometrium was collected from 40 normal cycling or from an equal number of women with histologically proven endometriosis, by endometrial biopsy under an approved institutional Human Investigations Committee protocol. Half of the tissue was immediately frozen in the liquid nitrogen and stored at −72° C. The other half of the tissue sample was fixed in formalin, embedded in paraffin, sectioned and stained with haematoxylin and eosin. Menstrual cycle dating was determined by menstrual history and confirmed by histological examination using the criteria of Noyes et al. (1950). Patients receiving hormonal therapy or currently undergoing evaluation of infertility were excluded.

Tissues were individually homogenized in 4 M guanidinium thiocyanate, 25 mM sodium citrate (pH 7.0), 0.5% sarkosyl, and 0.1 M 2-mercaptoethanol. Total RNA was size-fractioned on a 1% agarose 0.66 M formaldehyde gel and sequentially hybridized with a P-labeled riboprobe as described below. Hybridization was performed overnight at 60° C. in 50% formamide, 1× sodium chloride/sodium citrate (SSC), 5× Denhardt's reagent, 0.2% tRNA, and P-labeled riboprobe at 2×10⁶ cpm/ml. The filter was washed twice at 86° C. for 30 min in 0.1× SSC and 0.1% SDS. Kodak (Rochester, N.Y., USA) X-Omat AR film was exposed overnight at −70° C.

Plasmids used for probe preparation were a generous gift from E. Boncinnelli. pGEM plasmids containing sequence from the 3′ untranslated region of either human HOXA10 or HOXA11 were linearized with EcoRI or HindIII (New England Biolabs, Beverly, Mass., USA), ethanol precipitated and used as a template for generation of riboprobes. Radiolabeled RNA probes were generated by in-vitro transcription using the Promega Riboprobe Kit (Promega, Madison, Wis., USA). Antisense probes were generated using the appropriate RNA polymerase (T7 or SP6) and labeled with α-[P]-UTP (Amersham, Arlington Heights, Ill., USA).

The autoradiographic bands were quantified using a laser densitometer (Molecular Dynamics Inc., Sunnyvale, Calif., USA). Each HOXA10 or HOXA11 band was normalized to the value obtained from the same lane hybridized to glyceraldehyde-3-phosphate dehydrogenase (GAPDH). Data were analyzed using analysis of variance. P<0.05 was considered to be statistically significant.

Endometrium from 40 control patients was analyzed for HOXA10 and HOXA11 expression by Northern blot analysis. In all patients, the expected up-regulation of HOXA10 and HOXA11 expression in the mid-luteal phase was observed, as we have previously demonstrated (Taylor et al., 1998, 1999).

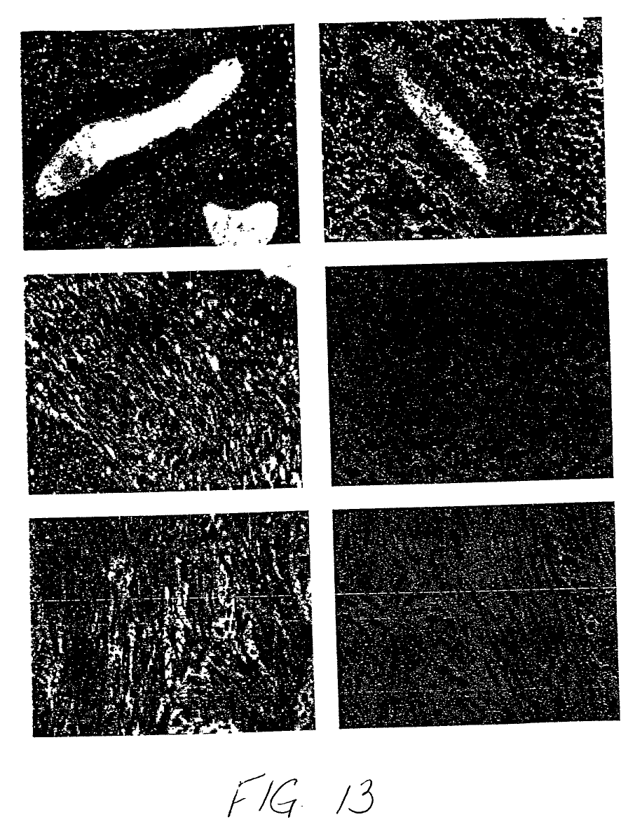

Endometria from 40 patients with endometriosis were analyzed in the same fashion. The eutopic endometria obtained from patients with endometriosis failed to show the equivalent up-regulation of either the HOXA10 or HOXA11 genes at the time of implantation. Representative samples are shown in FIG. 9. Densitometry was performed on the Northern blot analysis of all samples normalized to GAPDH, and summarized in FIG. 10. Levels of HOXA10 expression were similar in the proliferative and early secretory phases and not statistically different. In both the mid- and late segments of the secretory phase, a statistically significant difference was noted in endometrial HOXA10 expression between patients with or without endometriosis (P<0.01). A similar difference was noted with HOXA11 (FIG. 11). This failure to increase HOX gene mRNA levels did not depend on the stage of the disease and occurred despite in-phase endometrial histology.

The pathogenesis of endometriosis associated infertility is unclear. These data suggest a defect in regulation of HOX gene expression in the endometrium of patients with endometriosis. Expression of each of these genes is necessary for implantation. Failure of the normal increase in HOXA10 and HOXA11 mRNA levels to occur at the beginning of the window of implantation, may be one mechanism responsible for endometriosis related infertility. Whether this defect is inherent to the eutopic endometrium or the result of other factors associated with endometriosis remains to be demonstrated. These data suggest that a defect in endometrial development exists in patients with endometriosis, and that failure of implantation may contribute to their infertility.

Alterations in other molecules expressed in the endometria have been reported in endometriosis (Isaacson et al., 1990; Lessey et al, 1994; Noble et al., 1996; Shifren et al., 1996; Fujimoto et al., 1997; Sharpe Timms, 1997; Sillem et al., 1997). In the absence of histological alteration, molecular defects in the endometrium may be responsible for failure of implantation. In mice with a targeted disruption of either the Hoxa10 or Hoxa11 gene, implantation cannot occur despite a histologically normal endometrium (Hsieh-Li et al., 1995; Satokata et al., 1995). Similarly a defect in HOX expression in patients with endometriosis may lead to a decrease in implantation without an appreciable pathology noted on histological examination. Very few molecules are known to affect implantation specifically when a targeted mutation is produced in mice; it is interesting to note that these defects are often undetectable on histological examination. Molecular markers may be a more valuable way to assess the receptivity of the endometrium.

Hox genes function as transcription factors and are early regulators of tissue identity in embryonic development (Krumlauf, 1992; McGinnis and Krumlauf, 1992). It is likely they function in an analogous role in cyclic endometrial development (Taylor et al., 1998, 1999). Other molecular markers of implantation or of endometrial development are likely downstream target genes of Hox genes—either direct or indirect. It will be interesting to determine if any of the structural molecules or growth factors that are involved in implantation are regulated by HOXA10 or HOXA11. Alterations in HOX genes can be expected to produce a cascade of other defects in the expression of downstream target genes. Hox genes may be important early initiators of signal transfection that lead to the proper molecular development of the endometrium and to endometrial receptivity. It is likely many of the molecular, ultrastructural and clinical alterations seen in patients with endometriosis are mediated through alterations in HOX gene expression.

In further accordance with the invention, successful gene transfection and expression has been accomplished in the intact human uterus.

Gene therapy has been used for correction of metabolic defects, in cancer and in infectious diseases. There has been no report of in vivo gene therapy to the female reproductive tract. We assessed the ability to transfect the intact human uterus ex vivo. The uterine lumen was accessed transcervically using an intrauterine insemination catheter. pcDNA 3.1 plasmid containing the LacZ reporter gene was delivered via liposome mediated transfection. Control, uteri were transfected with empty pcDNA3.1 vector. β-Galactosidase expression was evaluated by immunohistochemistry. β-Galactosidase expression was observed in the LacZ treated uteri in endometrial epithelial cells, stroma and adjacent myometrium. Highest expression was seen in endometrial glandular epithelial cells with significant expression in the stroma and adjacent myometrium. Successful gene transfection and expression in the intact human uterus can be accomplished easily, rapidly and efficiently. Gene therapy may have wide applicability in the treatment and study of gynecologic disease.

Diseases of the reproductive tract impact the lives of many women, often resulting in morbidity, hospitalization and surgery. Currently used medical and surgical treatments are invasive, expensive and often associated with systemic side effects. We evaluated the possibility of using targeted gene therapy to the human uterus as a novel, alternative therapeutic approach to gynecologic disease.

Gene therapy is an emerging treatment modality that has the potential to substantially advance the limits of current therapeutics. With targeted disease and organ-specific administration, it is possible to precisely treat a condition and limit therapy to the affected area, thereby minimizing adverse therapeutic reactions. Gene therapy trials so far have focused on treatment of diseases caused by inherited or acquired genetic defects such as cystic fibrosis, alpha₁-antitrypsin deficiency and cancer or for the treatment of infections. As the genetic basis of many diseases and physiological states is better understood, the potential for application of gene therapy increases.

The molecular basis of uterine diseases such as leiomyomas, polyps, implantation defects, hyperplasia and cancer are under extensive investigation. This has led to a greater understanding of the underlying pathogenesis. Correction of molecular defects may be possible with gene therapy, offering a new approach to the diagnosis and treatment of uterine disease. By altering endometrial receptivity, gene therapy to the endometrium also has applications in the fields of infertility and contraception.

Due to the ease of access, the uterus is an ideal organ for gene therapy. Successful transfer is possible with an intrauterine insemination device in an office setting. Lack of need for cervical dilation minimizes discomfort and is well tolerated by most patients. Successful transfection of human endometrial epithelial cells in-vitro has been described using liposomes. Endometrium can also be obtained easily for evaluation of the results of gene therapy with an outpatient endometrial biopsy.

The mechanism of administration of gene therapy predominantly entails the use of viral vectors or liposomes. A major disadvantage of using viral vectors is systemic dissemination and immunogenicity. These have lead to complications, limiting the usefulness of gene therapy. Liposomes have traditionally been associated with low intracellular gene transfer efficiency. However, cationic liposomes have overcome this deficiency and have been evaluated as an alternative method of gene delivery. Liposomes are effective for local transfection and have a low likelihood of systemic dissemination. The present study was performed to assess the feasibility of application of liposome mediated gene therapy to the uterus in a safe, effective and reproducible manner.

In order to determine the feasibility of using gene therapy in the female reproductive tract, we performed ex-vivo transfection. The intact human uterus was instilled with a pcDNA3.1/LacZ/liposome mixture using an intrauterine insemination catheter. The LacZ gene encodes E. Coli β-galactosidase, which is not endogenously expressed in humans. Control uteri were transfected with empty pcDNA3.1 vector/liposome using the same protocol. The uteri were incubated in enriched Minimum Essential Medium (GIBCOBRL) on ice for 5 hours. The uterine tissue was fixed in formalin, paraffin embedded and sectioned. Using a mouse Anti-β Galactosidase IgM mouse antibody, expression of β-galactosidase was assessed by Immunohistochemistry. The uterine tissues were evaluated for efficacy as well as cellular specificity of transfection.

FIG. 12 demonstrates a low power photomicrograph of the results of immunohistochemistry. Abundant β-galactosidase expression is seen in multiple cell-types indicative of successful transfection. β-galactosidase expression is observed in the endometrium through its full thickness. In addition the myometrium demonstrates β-galactosidase expression to a depth of 1.75 cm from the endometrial-myometrial junction. Highest expression is seen in the surface and glandular epithelium. Significant but lower expression is seen in the endometrial stroma and adjacent myometrium. In the control uterus, transfected with the empty pcDNA 3.1 vector, no β-galactosidase expression is seen.

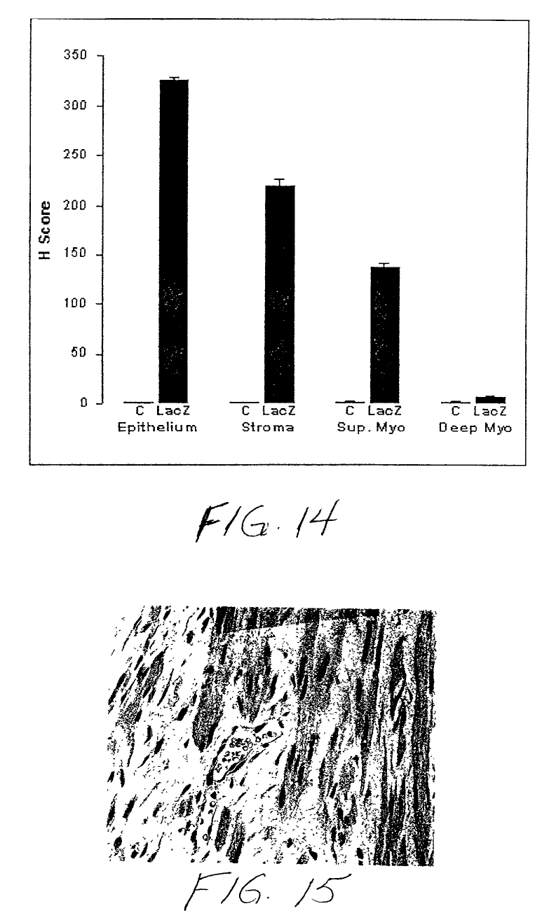

Cellular specificity of expression is demonstrated under high power in FIG. 13. We obtained β-galactosidase expression in the endometrial glandular epithelium, endometrial stroma and myometrium in the pcDNA3.1/LacZ transfected uterus. No expression was seen in the control uterus. β-Galactosidase expression is maximal in the endometrial glandular epithelium. In this tissue all cells display significant expression and 50% display dense staining. Similarly all stromal cells also display significant expression with 20% staining intensely. A gradient of β-galactosidase expression is seen in the myometrium. Shown in FIG. 13 is a representative section of myometrium 0.25 cm from the endometrial-myometrial junction where all cells display staining, but only 15% of cells show intense staining.

Analysis of H-Scores (a semi-quantitative method that incorporates both the intensity and distribution of staining) for individual cell components (FIG. 14) reveals that the intensity of staining for each is significantly different from respective controls at p<0.0001. The endometrial epithelial cells demonstrate significantly greater β-galactosidase expression (328±5) than either stroma (220±6), superficial (138±4) or distal (7±1) myometrium p<0.0001). The endometrial stromal cells also exhibit significantly greater β-galactosidase expression than either the adjacent myometrium or the distal myometrium (p<0.0001).

Beyond a depth of 1.75 cm from the endometrial-myometrial junction, no β-galactosidase expression is seen. Additionally, no peri- or intravascular expression of β-galactosidase was observed indicating that the liposome mediated transfection was contained within the intended target tissue (FIG. 15).

The success of gene therapy depends not only on successful transfection, but on eventual expression of the protein that the gene encodes. The bacterial LacZ gene encodes the enzyme β-galactosidase. In this experiment, we perfused the uterine cavity with a pcDNA3.1/LacZ-liposome mixture in order to transfect the intact human uterus. We examined the endometrium for expression of β-galactosidase. β-galactosidase is abundantly expressed in all cells of the endometrial glandular epithelium of treated uteri and to a significant extent in the stroma. The superficial myometrium showed lower but significant expression. The uteri treated with pcDNA3.1 alone failed to demonstrate any β-galactosidase expression. The demonstration of β-galactosidase expression in the LacZ treated uteri indicate that the processes of gene transfection, transcription and translation all occurred successfully.

The level of β-galactosidase expression was highest in the endometrial glandular epithelium. High levels of expression in the epithelium may occur because the liposome-DNA mixture is installed into the endometrial cavity where it is in intimate contact with the epithelial cells. The cell culture medium, instilled into the uterine cavity along with the DNA/liposome, may ensure better survival of glandular epithelial cells ex-vivo than either the stroma or myometrium and promote epithelial cell activity leading to greater protein synthesis. Alternatively, different cell-types may have inherent differences in the ability to process transfected genes. Epithelial cells may have an enhanced ability to express transfected genes over that of other uterine cell types. The stroma and myometrium, which are exposed to the gene-liposome mixture only indirectly due to flux across more superficial layers, demonstrate lesser expression.

An advantage of liposome mediated transfection over the use of viral vectors as the vehicle for gene therapy is that transfection is confined to the treatment area with a low likelihood of systemic spread. Dissemination of viral vector can result in complications such as viral infection, ectopic gene expression and host immune response. Using liposome mediated gene transfection, we found that maximal expression of β-galactosidase is obtained in the endometrial epithelium with no expression beyond 1.75 cm of the endometrial-myometrial junction. β-galactosidase expression is also absent in blood vessels. Treatment of endometrial disease by this method is therefore likely to lead to minimal untoward systemic effects.

The uterus is a readily accessible organ for gene therapy. Intrauterine gene therapy can be performed as an out-patient, requires no anesthesia and is well tolerated. Our results demonstrate that the treatment is effective locally with minimal spread and with an early onset of action (within 5 hours). Gene therapy may therefore be most useful in the endometrial epithelial conditions such as hyperplasia, dysplasia and cancer as well as in contraception. Due to significant stromal expression, gene therapy may also be useful in epithelial-stromal conditions such as implantation defects. This method of gene transfection does not result in abundant myometrial gene expression and may be less useful in treating myometrial disease.

Gene therapy to the female reproductive tract is feasible. It can be accomplished easily, rapidly and efficiently and is likely to be effective in a number of gynecological conditions.

Four patients undergoing abdominal hysterectomy for intractable chronic pelvic pain that had failed medical management provided uteri under an approved HIC Protocol. The patients were parous with an average age of 37 years (range 35-39 years). Two uteri were included in the study group and the other two used as controls. Histopathological evaluation revealed no evidence of uterine pathology. All patients were in the proliferative phase of the menstrual cycle.

pcDNA3.1(+)/LacZ (Invitrogen) vector was combined with cationic liposome and used for transfection. The liposome consisted of a 3:1 (w/w) formulation of 2,3-dioleyloxy-N-[2(sperminecarboxamido)ethyl]-N—N-dimethyl-1-propanaminium trifluoroacetate (DOPSA) and dioleoylphosphatidyl ethanolamine (DOPE) (LipofectAMINE; GIBCOBRL). Two μl of pcDNA/LacZ (8 μg/μl) in TE Buffer were mixed with 20 μl liposome (2 mg/ml) and incubated at room temperature (22° C.) for 15 minutes. At the end of the incubation period, 976 μl 1× Dulbecco's Phosphate Buffered Saline (PBS) was added to obtain a total volume of 1 ml. The final concentration of DNA was 16 μg/ml and liposome was 40 μg/ml in 1× PBS. We had previously achieved successful in-vitro transfection of a human endometrial cell line using this protocol.

After Hysterectomy, the uterus was immediately placed on ice and the uterine cavity was installed with 1 ml of the DNA/Liposome mixture in 1X PBS using an intrauterine insemination catheter (Wallach Surgical Devices Inc., USA). The uterus was incubated in Minimum Essential Cell Culture Medium (GibcoBRL) enriched with Sodium Pyruvate and 10% Fetal Bovine Serum for 5 hours. At the end of the incubation, the uterus was transferred to 10% Formalin and incubated overnight.

Cellular expression of β-galactosidase was evaluated by Immunohistochemistry using a mouse monoclonal IgM antibody (G6282: Sigma) developed against E. Coli β-galactosidase. Four random biopsies were obtained from each uterus. The specimens were embedded in paraffin and sectioned into serial 5 μm sections. The sections were deparaffinized in Xylene and Ethanol. Blocking of Endogenous Peroxidase was done by incubation with 0.6% H₂O₂. The sections were incubated with 1.5% normal goat blocking serum for 45 min. and then incubated overnight at 4° C. with the primary antibody (G6282: 4 μg/ml).

The slides were then incubated with biotinylated mouse secondary IgM antisera for 30 min at room temperature, followed by a 45 min incubation with Avidin and Biotinylated Peroxidase (Vectastain). The slides were then incubated in Diaminobenzidene (400 μg/ml) for 5 min. Counterstaining was performed with Hematoxylin and Li₂CO₃.

Analysis of immunohistochemical staining was performed using the H-Score. The semi-quantitative method incorporates both the intensity and distribution of staining. The intensity of staining was graded using an arbitrary scale from 0 to 3. Staining is characterized as no staining (0), weak but detectable (1), distinct (2) or very strong (3). The endometrial glandular epithelium, endometrial stroma and myometrium were analyzed for the percentage of cells in each staining category to obtain an aggregate H-Score for each cell type:

H-Score=ΣPᵢ(i+1)

Where Pᵢ is the percentage of stained cells in each intensity category, and i is the intensity score (1, 2, 3). Analysis was performed by three independent blinded observers.

The Mann-Whitney Test for Non-parametric variables was used to analyze the H-Scores in cell types between the pcDNA3.1/LacZ and control pcDNA3.1 treated uteri. ANOVA was used to evaluate the expression scores among the various cellular compartments in the LacZ treated uteri.

What is claimed is:

1. A method for treating the uterus comprising transfecting the uterus with a liposome-mediated gene.

2. The method of claim 1, wherein said liposome-mediated gene alters expression of human endometrial HOXA10.

3. The method of claim 1, wherein said liposome-mediated gene is a homeobox gene.

4. The method of claim 1, wherein said liposome-mediated gene is prepared using cationic liposome.
